(19) United States
(12) Patent Application Publication
(10) Pub. No.: US 2019/0125569 A1
(43) Pub. Date: May 2, 2019

(54) GARMENT WITH AN INTEGRATED URINE RESERVOIR

(71) Applicant: Charlie Dunham, St. Stephens, AL (US)

(72) Inventor: Charlie Dunham, St. Stephens, AL (US)

(21) Appl. No.: 15/801,634

(22) Filed: Nov. 2, 2017

(51) Int. Cl. A61F 5/44; A41D 1/06; A41D 27/02; A41D 31/02

(52) U.S. Cl. CPC A61F 5/4404; A61F 5/4408; A61F 5/4407; A41D 2600/00; A41D 27/02; A41D 31/02; A41D 2400/48; A41D 1/06

(57) ABSTRACT

A garment with an integrated urine reservoir for receiving discharged urine includes an outer layer that forms a pair of pants. The pair of pants includes a crotch portion, a right leg portion and a left leg portion. The outer layer has an inner surface and an outer surface. The outer layer comprises a cloth material. An inner liner is positioned within the outer layer. The inner liner forms a crotch section, a right leg section and a left leg section such that the inner liner defines an inner pair of pants and the outer layer defines an outer pair of pants. The inner liner comprises a fluid impermeable material. The fluid impermeable material is a flexible plastic material. The right leg section and the left leg section each comprise reservoirs to receive and hold urine.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to urine collecting garment devices and more particularly pertains to a new urine collecting garment device for receiving discharged urine.

### BRIEF SUMMARY OF THE INVENTION

An embodiment of the disclosure meets the needs presented above by generally comprising an outer layer that forms a pair of pants. The pair of pants includes a crotch portion, a right leg portion and a left leg portion. The outer layer has an inner surface and an outer surface and comprises a cloth material. An inner liner is positioned within the outer layer. The inner liner forms a crotch section, a right leg section and a left leg section such that the inner liner defines an inner pair of pants and the outer layer defines an outer pair of pants. The inner liner comprises a fluid impermeable material. The fluid impermeable material is a flexible plastic material. The right leg section and the left leg section each comprise reservoirs to receive and hold urine.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front side view of a garment with an integrated urine reservoir according to an embodiment of the disclosure.

FIG. 2 is a front view of an embodiment of the disclosure.

FIG. 3 is a top view of an embodiment of the disclosure.

FIG. 4 is a cross-sectional view of an embodiment of the disclosure taken along line 4-4 of FIG. 3.

FIG. 5 is a cross-sectional view of an embodiment of the disclosure taken along line 5-5 of FIG. 2.

### DETAILED DESCRIPTION OF THE INVENTION

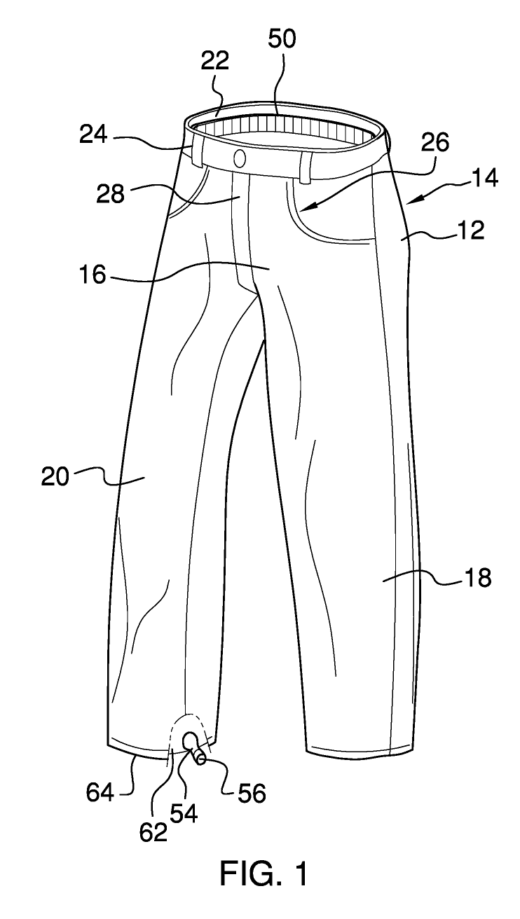

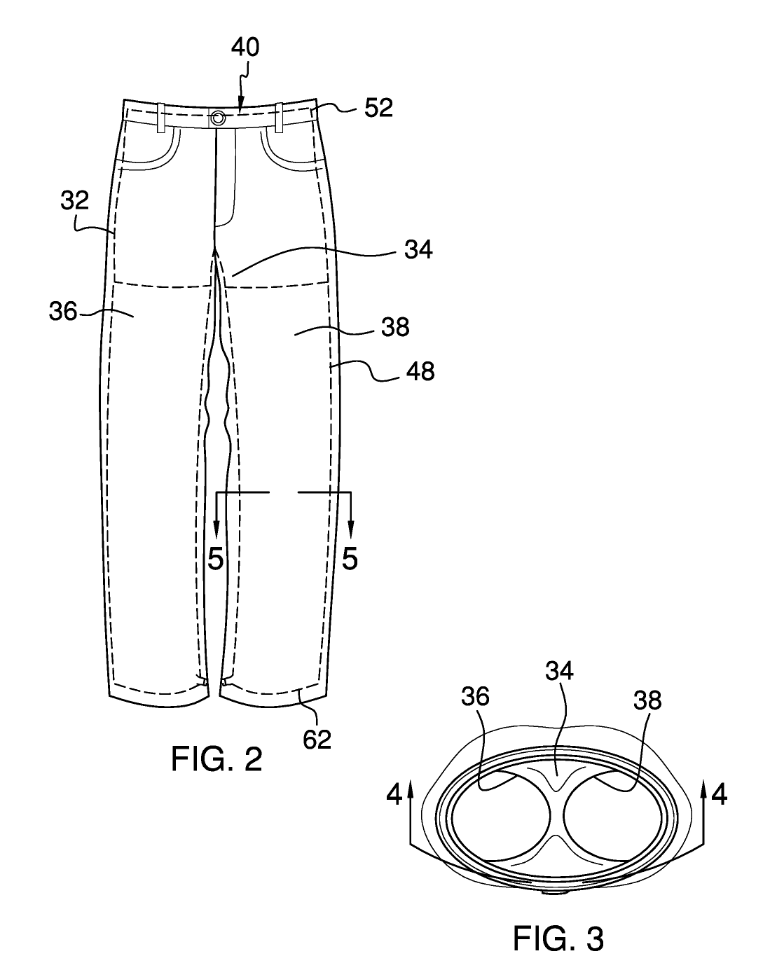

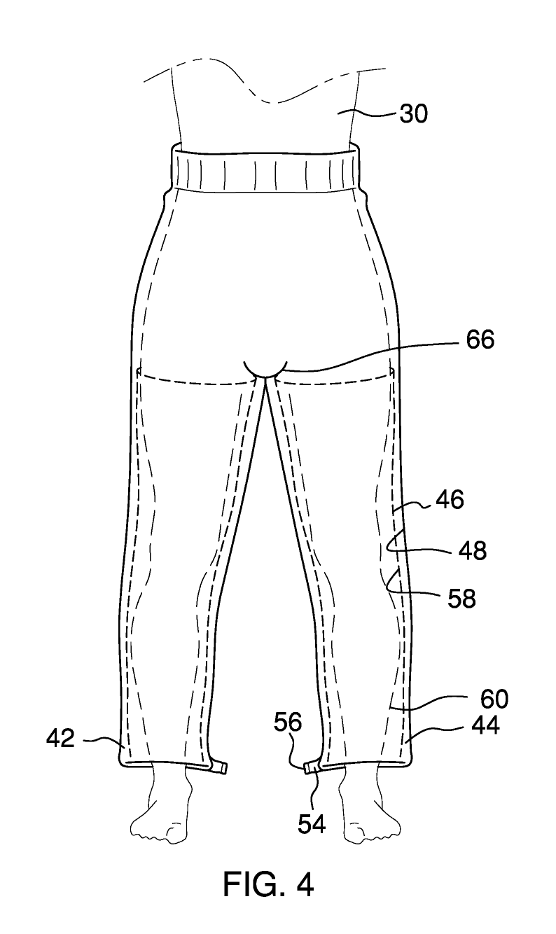

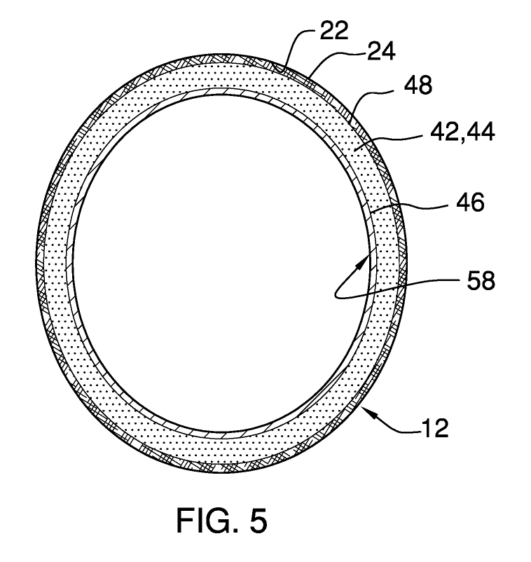

With reference now to the drawings, and in particular to FIGS. 1 through 5 thereof, a new urine collecting garment device embodying the principles and concepts of an embodiment of the disclosure and generally designated by the reference numeral 10 will be described.

As best illustrated in FIGS. 1 through 5, the garment with an integrated urine reservoir 10 generally comprises an outer layer 12 that forms a pair of pants 14. The pair of pants 14 includes a crotch portion 16, a right leg portion 18 and a left leg portion 20. The outer layer 12 has an inner surface 22, an outer surface 24. The outer layer 12 comprises a cloth material and may include a plurality of pockets 26 that is positioned on the outer surface 24. A fastener 28 may be positioned adjacent to the crotch portion. The fastener may be a zipper, button-fly or other types of fastener suitable to fasten the pair of pants 14 to a person 30.

An inner liner 32 is positioned within the outer layer 12 and forms a crotch section 34, a right leg section 36 and a left leg section 38 such that the inner liner 32 defines an inner pair of pants 40 and the outer layer 12 defines an outer pair of pants 14. The inner liner 32 comprises of a fluid impermeable material. The fluid impermeable material may be a flexible plastic material or any other material that does not allow fluid to pass through.

The right leg section 36 and the left leg section 38 each comprise reservoirs 42, 44 that receive and hold urine. Each of the right and left leg sections 36, 38 include an interior wall 46 and an exterior wall 48 such that the reservoirs 42, 44 are positioned between the interior 46 and the exterior wall 48. The exterior wall 48 extends upwardly from the right and left leg sections 36, 38 and forms the crotch section 34 such that the crotch section 34 is in fluid communication with the reservoirs 42, 44. A band 50 is attached to and extends along an upper peripheral edge 52 of the crotch portion 34. The band 50 comprises a resiliently stretchable material. A pair of drain conduits 54 is included wherein each of the right and left leg sections 36, 38 has one of the drain conduits 54 fluidly coupled thereto. Each of the drain conduits 54 includes a closure 56 that is openable to release fluid from a corresponding one of the reservoirs 42, 44.

An inner layer 58 is positioned on the interior wall 46 of each of the right and left leg sections 36, 38 and abuts against a skin 60 of a person 30. The inner layer 58 comprises a cloth material. The inner layer 58 has a bottom edge 62 that is attached to a lower edge 64 of the outer layer 12.

In use, the assembly 10 is worn as a conventional pair of pants 14 wherein the crotch section 34 abuts a crotch of the person 30. Urine released by the person 30 falls downwardly into one of the reservoirs 42, 44. To empty the reservoirs 42, 44, the drain conduit 54 is positioned over a toilet and a corresponding one of the closures 56 is opened to release fluid outwardly from the drain conduit 54.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A urine collecting garment assembly configured for receiving discharged urine, said assembly comprising:
an outer layer forming a pair of pants including a crotch portion, a right leg portion and a left leg portion, said outer layer having an inner surface and an outer surface, said outer layer comprising a cloth material; and
an inner liner being positioned within said outer layer and forming a crotch section, a right leg section and a left leg section such that said inner liner defines an inner pair of pants and said outer layer defines an outer pair of pants, said inner liner being comprises of a fluid impermeable material, said fluid impermeable material being a flexible plastic material, said right leg section and said left leg section each comprising reservoirs for receiving and holding urine.

2. The urine collecting garment assembly according to claim 1, said right leg section and said left leg section each comprising reservoirs for receiving and holding urine, each of said right and left leg sections including an interior wall and an exterior wall such that said reservoirs are positioned between said interior and said exterior walls, said exterior wall extending upwardly from said right and left leg sections and forming said crotch section such that said crotch section is in fluid communication with said reservoirs.

3. The urine collecting garment assembly according to claim 1, further including a band being attached to and extending along an upper peripheral edge of said crotch portion, said band being comprised of resiliently stretchable material.

4. The urine collecting garment assembly according to claim 1, further including a pair of drain conduits, each of said right and left leg sections having one of said drain conduits fluidly coupled thereto, each of said drain conduits including a closure being openable to release fluid from a corresponding one of said reservoirs.

5. The urine collecting garment assembly according to claim 2, further including a pair of drain conduits, each of said right and left leg sections having one of said drain conduits fluidly coupled thereto, each of said drain conduits including a closure being openable to release fluid from a corresponding one of said reservoirs.

6. The urine collecting garment assembly according to claim 2, further including an inner layer being positioned on said interior wall of each of said right and left leg sections and being configured to be abutted against a skin of a person, said inner layer comprising a cloth material, said inner layer having a bottom edge being attached to a lower edge of said outer layer.

7. A urine collecting garment assembly configured for receiving discharged urine, said assembly comprising:
an outer layer forming a pair of pants including a crotch portion, a right leg portion and a left leg portion, said outer layer having an inner surface and an outer surface, said outer layer comprising a cloth material;
an inner liner being positioned within said outer layer and forming a crotch section, a right leg section and a left leg section such that said inner liner defines an inner pair of pants and said outer layer defines an outer pair of pants, said inner liner being comprises of a fluid impermeable material, said fluid impermeable material being a flexible plastic material;
said right leg section and said left leg section each comprising reservoirs for receiving and holding urine, each of said right and left leg sections including an interior wall and an exterior wall such that said reservoirs are positioned between said interior and said exterior walls, said exterior wall extending upwardly from said right and left leg sections and forming said crotch section such that said crotch section is in fluid communication with said reservoirs;
a band being attached to and extending along an upper peripheral edge of said crotch portion, said band being comprised of resiliently stretchable material;
a pair of drain conduits, each of said right and left leg sections having one of said drain conduits fluidly coupled thereto, each of said drain conduits including a closure being openable to release fluid from a corresponding one of said reservoirs; and
an inner layer being positioned on said interior wall of each of said right and left leg sections and being configured to be abutted against a skin of a person, said inner layer comprising a cloth material, said inner layer having a bottom edge being attached to a lower edge of said outer layer.
